(19) United States
(12) Patent Application Publication
(10) Pub. No.: US 2017/0319063 A1
(43) Pub. Date: Nov. 9, 2017

(54) APPARATUS AND METHOD FOR RECORDING AND ANALYSING LAPSES IN MEMORY AND FUNCTION

(71) Applicant: NeuroVision Imaging LLC, Sacramento, CA (US)

(72) Inventors: Steven Verdooner, Sacramento, CA (US); Rodney Sparks, Antelope, CA (US)

(21) Appl. No.: 15/590,235

(22) Filed: May 9, 2017

Related U.S. Application Data
(60) Provisional application No. 62/333,542, filed on May 9, 2016.

(51) Int. Cl. A61B 5/00; A61B 5/11; A61B 5/048; G06F 17/00; A61B 5/02

(52) U.S. Cl. CPC A61B 5/00; G06F 17/00; A61B 5/7264; A61B 5/02; A61B 5/11; A61B 5/048; A61B 5/4809

(57) ABSTRACT

An apparatus and method for sensing, recording and analyzing data representative events of memory lapses and function uses a wearable device (e.g., wrist, armband, pendant) having sensors to detect user gestures and vital signs for transmission and analysis by a computation unit to predict the onset of cognitive impairment related diseases.

### REFERENCE TO RELATED APPLICATIONS

The present application claims priority on and incorporates by reference U.S. provisional application Ser. No. 62/333,542 filed May 9, 2016.

### BACKGROUND OF THE INVENTION

The present invention relates to an apparatus and method for recording and analyzing lapses in memory and function using a wearable device.

There is no blood test or definitive way to diagnose Alzheimer's disease. An autopsy can provide a diagnosis, because the brain of someone with dementia has physical signs of the disease. Doctors rely on a battery of cognitive tests to diagnose Mild Cognitive Impairment (MCI) and Alzheimer's disease (AD). The neuropsychological battery of tests that are given to patients at different stages are subject to bias, are not very repeatable, do not account for environmental factors (such as a poor night's sleep, or taking the test with low blood sugar). These tests have severe limitations, especially in early stages of the disease. There also is no test that has excellent sensitivity and reproducibility for studying disease progression or response to therapy. Studies indicate that doctors should pay closer attention to self-reported memory complaints from their older patients. There is some agreement in the community that self-reporting, albeit subjective, is a reasonable way to determine if the condition is getting worse. Subjective memory complaints (SMC) are self identified deficits in memory. They are common among adults age 60+. (Nurses Health Study 56.4%, PREADVISE Study 22%)

According to researchers at the University of Kentucky, people who report memory complaints are at a higher risk of future cognitive impairment and have higher levels of Alzheimer-type brain pathology even when impairment does not occur. One of the conclusions is that physicians should query and monitor subjective memory complaints (SMC) in their older patients.

The research by the scientists at the University of Kentucky's Sanders-Brown Center on Aging suggests that people who notice their memory is slipping may be at risk for Alzheimer's disease.

The research, led by Richard Kryscio, PhD, Chairman of the Department of Biostatistics and Associate Director of the Alzheimer's Disease Center at the University of Kentucky, appears to confirm that self-reported memory complaints are strong predictors of clinical memory impairment later in life.

Kryscio and his group asked 531 people with an average age of 73 and free of dementia if they had noticed any changes in their memory in the prior year. The participants were also given annual memory and thinking tests for an average of 10 years. After death, participants' brains were examined for evidence of Alzheimer's disease.

During the study, 56 percent of the participants reported changes in their memory, at an average age of 82. The study found that participants who reported changes in their memory were nearly three times more likely to develop memory and thinking problems. About one in six participants developed dementia during the study, and 80 percent of those first reported memory changes.

“What's notable about our study is the time it took for the transition from self-reported memory complaint to dementia or clinical impairment—about 12 years for dementia and nine years for clinical impairment—after the memory complaints began,” Kryscio said. “That suggests that there may be a significant window of opportunity for intervention before a diagnosable problem shows up.”

Kryscio points out that while these findings add to a growing body of evidence that self-reported memory complaints can be predictive of cognitive impairment later in life, there isn't cause for immediate alarm if you can't remember where you left your keys.

“Certainly, someone with memory issues should report it to their doctor so they can be followed. Unfortunately, however, we do not yet have preventative therapies for Alzheimer's disease or other illnesses that cause memory problems. Reference: Neurology 2014; 83:1359-1365

Researchers watched 531 people over 10 years at the University of Kentucky. The participants were considered “cognitively intact” when they were enrolled. Each year, scientists asked them if they felt any changes in their memory since their last visit to the doctor's office. They did autopsies on participants who died to see if their brains showed physical signs of dementia. More than half the people enrolled in the study (55.7%) reported some memory complaints. Scientists found that those who reported struggling to remember things were more likely to have dementia down the road than those who did not report memory troubles. Mild cognitive impairment on average happened about 9.2 years after participants first noticed a problem.

The findings in this report are subject to some limitations as the results are based on a simple annual subjective question.

### SUMMARY OF THE INVENTION

There is a need for an apparatus and method to turn subjective questions and self reported observations relating to lapses in memory and function into objective measurements.

To accomplish this, the invention provides an apparatus in the form of wearable technology for users to self-report, record, document, and analyze lapses in memory and function, and in combination with environmental and other factors that can influence these results. The recorded data can be normalized against an age-matched normative database, and also to further adjust and account for sleep patterns, exercise, diet, heart rate, perspiration, and mobility patterns. Parts or all of this data from wearable technology would be combined to improve monitor progression and to improve predictive power.

Recording of lapses in memory and/or function can be accomplished in a number of ways on a wearable device. The first would allow a simple tap, or tap sequence on a wearable device. This could be accomplished by sensing a gesture, such as pressing a button on the wearable, or by tapping and creating a vibration that is detected by the accelerometer in the wearable “cognitive tap” (“COGTAP”). In another embodiment this could be accomplished by developing an applications program (app) that would allow the use of multiple brands of wearables and the ability to use the accelerometers in said wearables to record the time and date of these lapses based on a programmable tap sequence for that wearable that is indicative of a single or multiple types of impairments. In another embodiment the tap may only be used in the training step, thereby analyzing characteristics of other passive sensors that would be indicative of these lapses.

Incorporating this functionality into a proprietary (or any) wearable allows this data to be analyzed along with any combination of motion, mobility, heart rate, blood pressure, perspiration, and sleep patterns.

As one example, one could deduce that the frequency of these events increases in situations where sleep is sub-optimal or sleep-deprived. The COGTAP could be cross-correlated with and/or normalized to sleep and motion/mobility data. This data from multiple inputs from the wearable could be further combined into a combination risk factor score that incorporates elements of frequency of memory lapses, time and quality of sleep, amount and duration of exercise, mobility, heart rate, blood pressure, perspiration, and diet.

In another embodiment the COGTAP could initiate recording of audio so as to further analyze and understand the circumstances under which these lapses occurred and to determine the type of lapse (cognition or function, or sub-divided from there). This could be accomplished by a constant audio recording loop that in one embodiment would record the previous minute prior to the tap and also the minute post-tap. The audio would be continually streaming to a buffer but not save an audio recording event unless initiated. Same could be accomplished with both audio and video recording of the person and/or surrounding environment. In another embodiment, audio could be continuously recorded along with annotation of memory lapses (and other wearable data previously listed) for further analysis by experts, and also to utilize a speech recognition engine to look for patterns. Speech recognition could further segment and differentiate lapses in memory from lapses in function. This differentiation could be diagnostically important. In another embodiment, all of the above could be implemented in a training mode, all data from lapse events are analyzed, cross correlated with a specific pattern from the sensors, and then programmed for future automated passive detection.

The invention provides a wearable sensor device, for sensing and recording data representative of events of memory lapses and function, comprising: a wearable sensor device; at least one gesture sensor in the wearable sensor device capable of sensing a gesture by the wearer, the gesture being representative of events of memory lapses and function; at least one vital sign sensor for sensing at least one vital sign condition being experienced by a wearer of the device; a memory for storing gesture data representing the sensed data from the gesture sensor, and for storing vital sign data sensed by the vital sign sensor; wherein the gesture data and vital sign data is adapted for transmission to a computation unit for analyzing the gesture data and vital sign data, comparing it to a reference database of normative data of age-matched subjects and for producing diagnosis data which predicts onset of cognitive impairment related diseases.

The invention provides a method for sensing and recording data representative of events of memory lapses and function, comprising: providing a wearable sensor device worn by a subject; sensing a gesture by the wearer using a gesture sensor in the wearable sensor device, the gesture being representative of events of memory lapses and function; sensing at least one vital sign condition being experienced by a wearer of the device using a vital sign sensor in the wearable sensor device; storing vital sign gesture data representing the sensed data from the gesture sensor, and storing vital sign data sensed by the vital sign sensor; transmitting the gesture data and vital sign data to a computation unit; comparing the gesture data and vital sign data to a reference database of normative data of age-matched subjects; and producing diagnosis data which predicts onset of cognitive impairment related diseases of the subject.

The invention also provides a non-transitory storage medium for storing instructions for performing the method of: sensing and recording data representative of events of memory lapses and function, comprising: providing a wearable sensor device worn by a subject; sensing a gesture by the wearer using a gesture sensor in the wearable sensor device, the gesture being representative of events of memory lapses and function; sensing at least one vital sign condition being experienced by a wearer of the device using a vital sign sensor in the wearable sensor device; storing vital sign gesture data representing the sensed data from the gesture sensor, and storing vital sign data sensed by the vital sign sensor; transmitting the gesture data and vital sign data to a computation unit; comparing the gesture data and vital sign data to a reference database of normative data of age-matched subjects; and producing diagnosis data which predicts onset of cognitive impairment related diseases of the subject.

### BRIEF DESCRIPTION OF THE DRAWINGS

FIG. 1A shows a wrist wearable device according to the invention with a button;

FIG. 1B shows a wrist wearable device according to the invention with button and heart rate, perspiration and blood oxygen sensors;

FIG. 1C shows a wrist wearable device like that of FIG. 1A but without a button, and with camera and microphone;

FIG. 1D shows a wrist wearable device like that of FIG. 1B but without a button;

FIG. 2A shows a wearable device like that of FIG. 1A, but worn on an arm instead of wrist;

FIG. 2B shows a wearable device like that of FIG. 1B, but worn on an arm instead of wrist;

FIG. 2C shows a wearable device like that of FIG. 1C, but worn on an arm instead of wrist;

FIG. 2D shows a wearable device like that of FIG. 1D, but worn on an arm instead of wrist;

FIG. 3A shows a pendant type wearable device with button, camera and microphone;

FIG. 3B shows a pendant type wearable device like FIG. 3A, but without a button;

FIG. 4A shows a pendant type wearable device without button and EEG sensor;

FIG. 4B shows a pendant type wearable device with button and EEG sensor;

FIG. 4C shows a pendant type wearable device with earbud EEG sensor and without button;

FIG. 4D shows a pendant type wearable device with earbud EEG sensor and button;

FIGS. 5A-5P show an anatomical figure representing a wearer having different versions of the wearable devices including the four wrist types, the four armband types, the two pendant types and the four pendant and EEG types;

FIG. 6 shows a block diagram of a wearable device in communication wirelessly or wired in LAN with a Wi-Fi router and through internet to a cloud server, wherein the wearable device constantly streams logged data and events as they occur in real time over wireless LAN (Wi-Fi), and wherein the wearable device communicates directly with cloud server and uploads logged data;

FIG. 7 shows a block diagram like that of FIG. 6, but including a Bluetooth low energy (BLE) central device, which may be a charging base or mobile phone, and transmits logged events as they occur in real time; and

FIG. 8 shows a block diagram like that of FIG. 7 but wherein the charging base transmits logged data to charging base while charging and then uploads logged data in batches (not real time).

### DETAILED DESCRIPTION OF THE INVENTION

One or more embodiments of the invention will be described as exemplary, but the invention is not limited to these embodiments.

The invention provides a wearable sensor device, for sensing and recording data representative of events of memory lapses and function, comprising: a wearable sensor device; at least one gesture sensor in the wearable sensor device capable of sensing a gesture by the wearer, the gesture being representative of events of memory lapses and function; at least one vital sign sensor for sensing at least one vital sign condition being experienced by a wearer of the device; a memory for storing gesture data representing the sensed data from the gesture sensor, and for storing vital sign data sensed by the vital sign sensor; wherein the gesture data and vital sign data is adapted for transmission to a computation unit for analyzing the gesture data and vital sign data, comparing it to a reference database of normative data of age-matched subjects and for producing diagnosis data which predicts onset of cognitive impairment related diseases.

The gesture sensor may detect at least one of a tap, a tap sequence, an audio signal, a video signal, a hand gesture, a head movement gesture, an audible trigger, and an EEG trigger. The vital sign sensor may detect at least one of heart rate, blood pressure, perspiration, EEG temperature and blood oxygen level. The device may include at least one activity sensor for detecting at least one of sleep exercise, motion and mobility. The device may communicate the gesture and vital sign data to a cloud server. The device may communicate the gesture and vital sign data through a router to a cloud server. The device may communicate the gesture and vital sign data through a Bluetooth low energy (BLE) device to a cloud server. The device may communicate the gesture and vital sign data through a charging base to cloud server. The device may communicate the gesture and vital sign data through a charging base and router to a cloud server. The device may communicate the gesture and vital sign data continuously in real time.

The device may communicate the gesture and vital sign data in batches. The computation unit may calculate a risk factor score based on at least one of frequency of memory lapses, time and quality of sleep, amount and duration of exercise, mobility, heart rate, blood pressure, perspiration and diet. The computation unit may predict onset of cognitive impairment related diseased by analyzing the circumstances under which the memory lapses and function occurred, and determining the type of memory lapse, including one or more components of cognitive or function. The computation unit may predict onset by analyzing gesture and vital sign data for a time period offset in time from a gesture representative of a memory lapse event. The time period offset may include a time period which precedes a gesture representative of a memory lapse event. The time period offset may include a time period which is subsequent to a gesture representative of a memory lapse event. The sensor may be an audio sensor and the gesture data may be audio data. The sensor may be a video sensor and the gesture data may be video data of the subject wearing the wearable device. The computation unit may further include a speech recognition unit. The computation unit may receive gesture data and vital sign data from a plurality of users wearing a wearable device, and uses the combined data to generate population risk factors. The combined data may be used to generate population risk factors for advancing disease. The computation unit may compare the gesture and vital sign data to previously obtained baseline data.

The invention provides a method for sensing and recording data representative of events of memory lapses and function, comprising: providing a wearable sensor device worn by a subject; sensing a gesture by the wearer using a gesture sensor in the wearable sensor device, the gesture being representative of events of memory lapses and function; sensing at least one vital sign condition being experienced by a wearer of the device using a vital sign sensor in the wearable sensor device; storing vital sign gesture data representing the sensed data from the gesture sensor, and storing vital sign data sensed by the vital sign sensor; transmitting the gesture data and vital sign data to a computation unit; comparing the gesture data and vital sign data to a reference database of normative data of age-matched subjects; and producing diagnosis data which predicts onset of cognitive impairment related diseases of the subject.

The sensing step may detect at least one of a tap, a tap sequence, an audio signal, a video signal, a hand gesture, a head movement gesture, an audible trigger, and an EEG trigger. The vital sign sensor may detect at least one of heart rate, blood pressure, perspiration, EEG temperature and blood oxygen level. The method may detect at least one of sleep exercise, motion and mobility of the subject, and providing activity data. The method may include communicating the gesture and vital sign data to a cloud server. The device may communicate the gesture and vital sign data through a router to a cloud server. The device may communicate the gesture and vital sign data through a Bluetooth low energy (BLE) device to a cloud server. The device may communicate the gesture and vital sign data through a charging base to cloud server. The device may communicate the gesture and vital sign data through a charging base and router to a cloud server. The device may communicate the gesture and vital sign data continuously in real time. The device may communicate the gesture and vital sign data in batches. The computation unit may calculate a risk factor score based on at least one of frequency of memory lapses, time and quality of sleep, amount and duration of exercise, mobility, heart rate, blood pressure, perspiration and diet. The computation unit may predict onset of cognitive impairment related diseased by analyzing the circumstances under which the memory lapses and function occurred, and determining the type of memory lapse, including one or more components of cognitive or function. The method may include predicting onset by analyzing gesture and vital sign data for a time period offset in time from a gesture representative of a memory lapse event. The method may include analyzing gesture and vital sign data in a time period which precedes a gesture representative of a memory lapse event. The method may include analyzing gesture and vital sign data in a time period which is subsequent to a gesture representative of a memory lapse event. The gesture data may be at least one of audio data and video data of the subject wearing the wearable device. The computation unit may further include a speech recognition unit for recognizing speech. The method may include receiving gesture data and vital sign data from a plurality of users wearing a wearable device, and using the combined data to generate population risk factors. The method may include generating population risk factors for advancing disease. The method may include comparing the gesture and vital sign data to previously obtained baseline data.

The invention provides an apparatus and method of use of a wearable device to record time, data, and frequency of lapses in memory and/or function. This can be accomplished a number of different ways, the following of which are non-limiting examples.

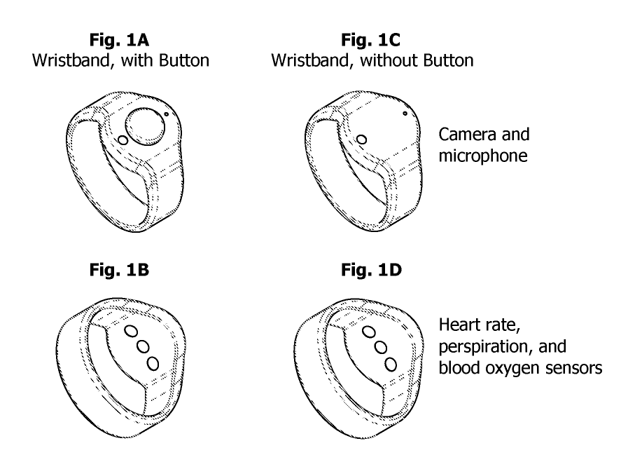

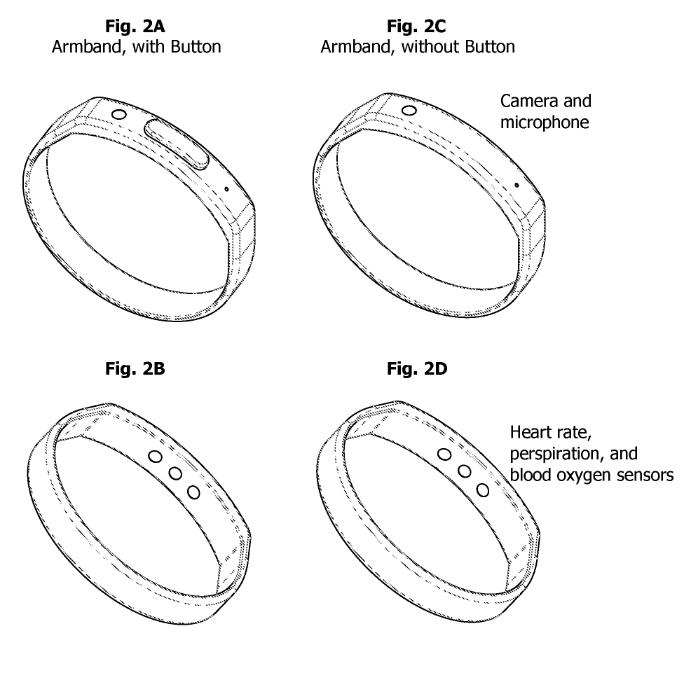

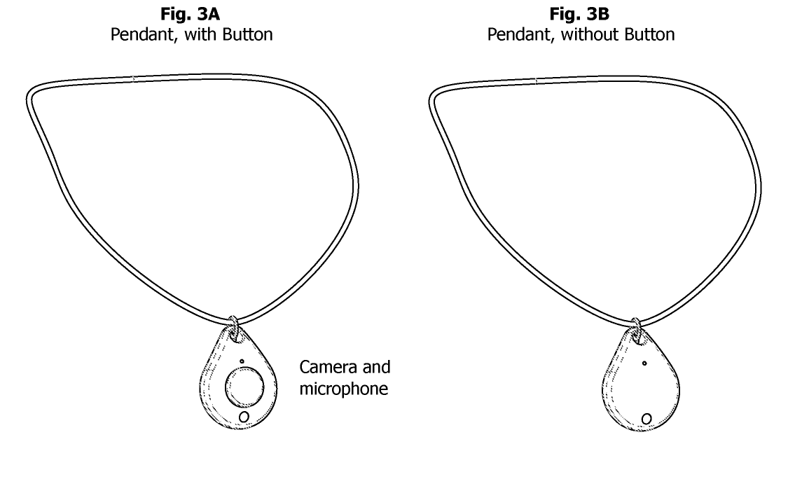

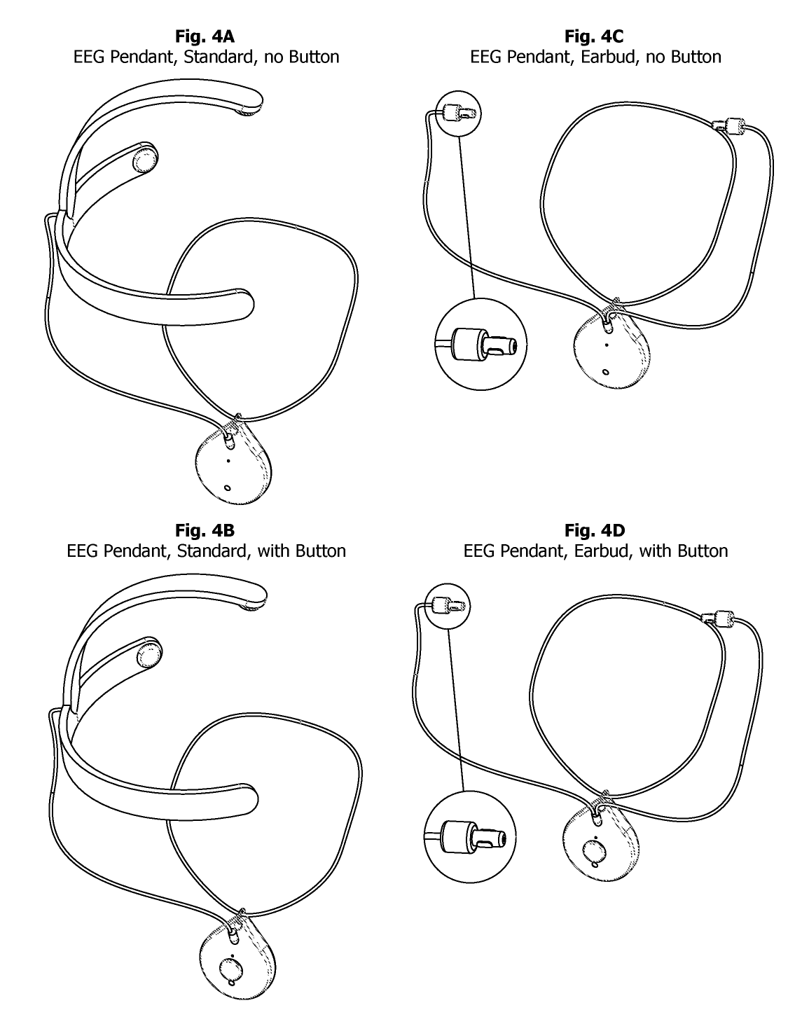

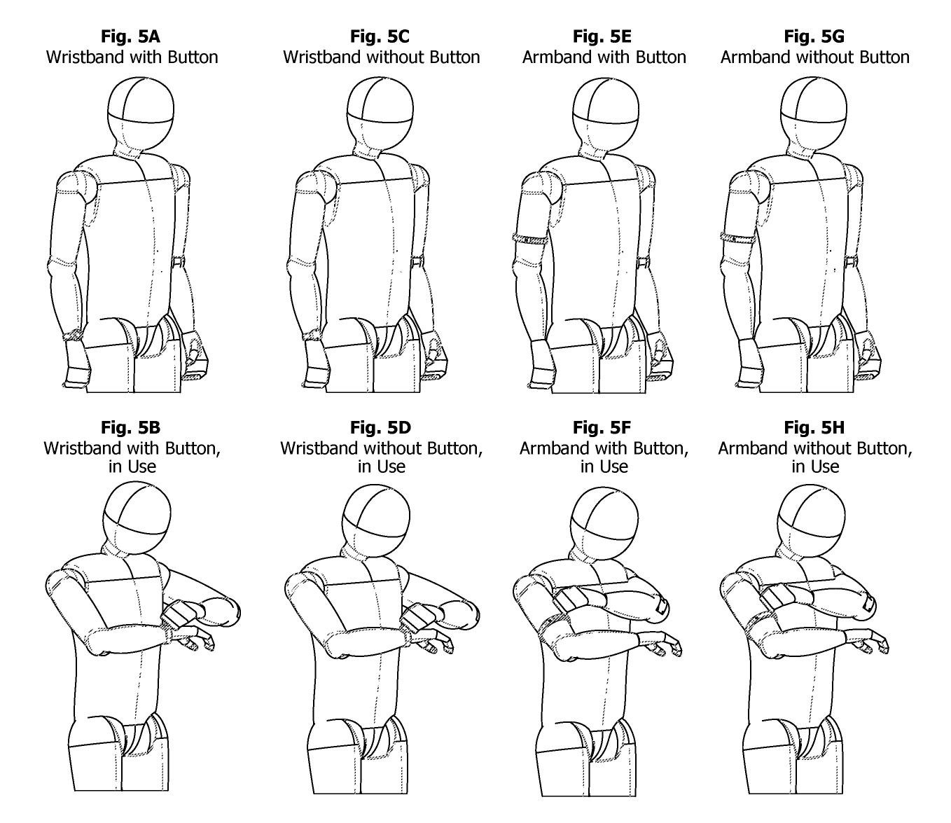

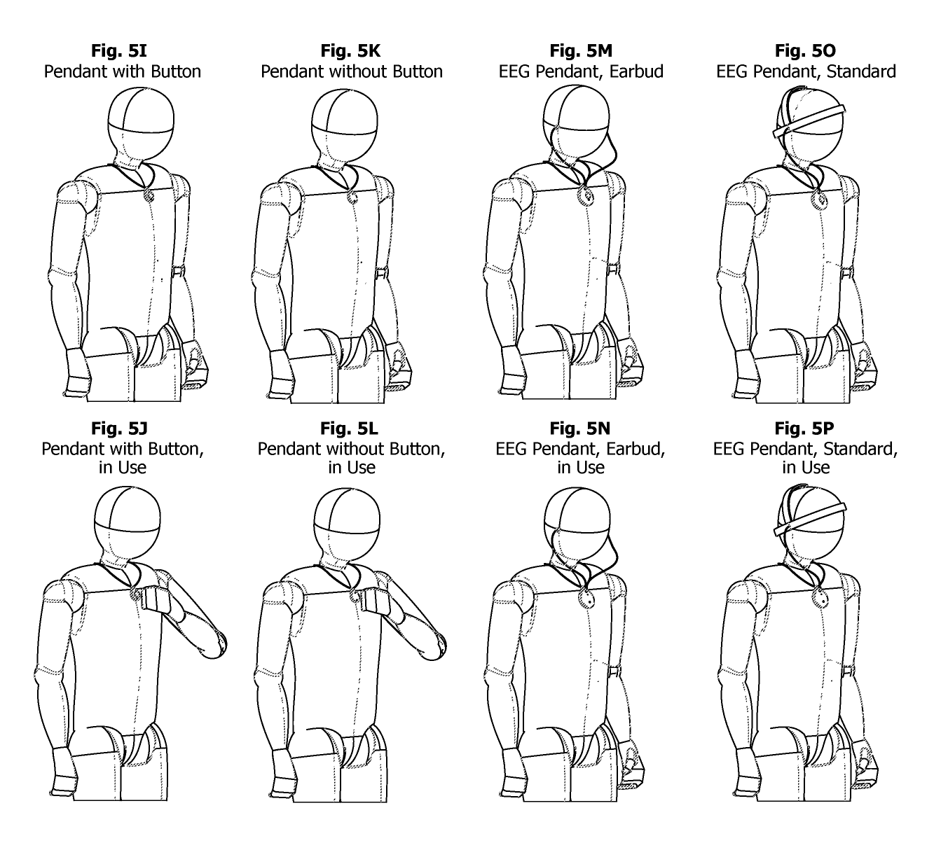

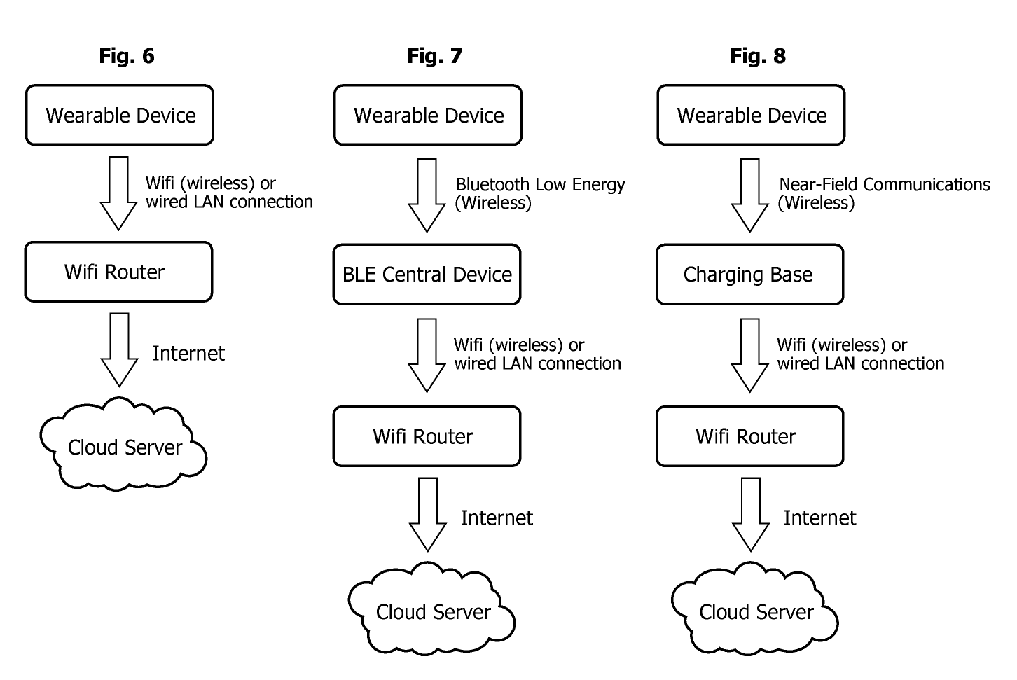

FIGS. 1A-1D show a wrist wearable device in different embodiments having different sensors, as described above in connection with the Drawing Figures. FIGS. 2A-2D show an arm wearable device in different embodiments having different sensors, as described above in connection with the Drawing Figures. FIGS. 3A and 3B show different type pendant wearable devices. FIGS. 4A-4D show a pendant type wearable device with an EEG sensor. FIGS. 5A-5P show an anatomical figure representing a wearer having the different versions of the wearable device. FIGS. 6, 7 and 8 show systems in which the wearable device can be used.

The wearable device can be responsive to a tap, multiple taps, tap pattern, tap pattern for each type of impairment, audio triggered with word recognition built into the wearable, audio recording for speech recognition of key words and phrases (no tap), gaze initiated (looking at a wearable with built in camera that is looking for visual ques or gestures, gesture based trigger with hand or head motion gestures, audible trigger (like a finger snap or other), EEG triggers via EEG devices (either traditional or earbud-born EEG sensor), or through a unique combination of sensors that are illustrative of a lapse event, either based on population training data, individual training data, or a combination thereof. This might also include vital sign data from advanced wearables that also include heart rate, blood pressure, perspiration monitor, and eeg, temperature, and other sensors, including environmental sensors not born on the wearable. Essentially a data signature from the unique combination of sensors triggers the recording of an event.

The use of this technology would be for patient selection for clinical trials, monitoring of healthy aging, monitoring of subjective memory complainer, MCI, or AD, measuring response to a lifestyle intervention program, supplement, therapy, or other intervention that could influence the measurement both positive and negative. The data could be combined with other biomarker and imaging data to better predict candidates for trials, onset of cognitive decline (MCI), AD, or to predict response to therapy or other intervention.

The invention provides a method of recording lapses in memory and/or function using varying ways of triggering a wearable to record and analyze said events. The frequency of these events could be analyzed and reported to the person or the doctor to indicate current status in a given time period and also to allow comparison over time to evaluate severity of situation, healthy aging progression, disease progression, or response to therapeutic treatment and/or lifestyle modification or intervention. If an audio recording is utilized, this could be combined with speech recognition to identify and patterns and differentiate different types of events and/or impairments. It may be important to differentiate memory impairment from functional impairment—this may be accomplished utilizing different types of tap codes, audio ques, gestures, combination of sensors, etc.

This data could be combined with other wearable obtained data (depending upon the wearable) such as: exercise, motion, mobility, heart rate, perspiration, blood pressure, eeg, and sleep data that is also generated by the wearable or combination of wearables and other sensors. A user could match their data against age/gender matched controls to further assess risk factors and generate a risk score. This could also be combined with other sensor data including but not limited to sleep, motion, mobility and other information to predict future onset of Mild Cognitive Impairment (MCI), Alzheimer's disease (AD), or other types of cognitive impairment. This apparatus and method could be utilized to measure response and efficacy of a therapeutic that is intended to slow or reverse cognitive decline. This method could be utilized to measure overall cognitive health and also in response to a lifestyle intervention program including diet exercise and dietary supplements.

In another embodiment, all the data is aggregated from multiple users to generate population based risk factors for advancing disease or to generate risk scores to report back to users and doctors.

In another embodiment, the lapses or other cognitive events are automatically recorded according to an algorithm that observes changes in mobility, heart rate, and perspiration (as compared to normal) as detected and automatically recorded by the wearable. This combination could be indicative of a stress event followed by patterns of sensors that indicate a lapse event. The time period of these sensor changes would be important to differentiate lapse events from other events that could trigger same sensor or sensor combination.

In another embodiment, all data from the wearable is recorded, uploaded to the cloud for post-processing, compared with deep learning big data set and analyzed for patterns consistent with memory and function lapses. In another embodiment, there is a training set for wearable obtained data that has previously been established using a tapping mechanism so as to generate a training set that consists of all the wearable parameters previously described. The training set could be population based, individual, or a combination thereof. This would provide the ability to assess triggers in the context of other wearable data. One could expect changes in a number of factors recorded by the wearable to be predictive of lapses and to be differentiated from other events. As an example, one might detect a change in heart rate and perspiration indicating a high level of stress for a specific period of time, combined with a sudden change in mobility while the user attempts to recall said memory. This pattern could potentially be identifiable based on analysis of multiple users, trained with multiple users, or simply trained by an individual user during a training period, or a combination thereof.

In one embodiment, a user could use a tap to indicate an event. One could then analyze multiple events from a user over a period of time (perhaps a month training period), generate the unique signal for that individual (as an example, increase heart rate and perspiration for x duration, followed by change in mobility, followed by a return to normal over a certain time period). One could utilize training data generated from numerous users to be predictive of an individual. One could then eliminate the need to tap for future events. One could utilize audio recording in the training set to better differentiate real events and types of events. Generally one could utilize the tap method alongside multiple wearable sensors or wearable EEG sensor to create a “training” set for a given patient, then utilize that data to automatically trigger (without a TAP) based on one or more of the wearable sensors (possibly including EEG data), and/or patterns or combinations of the wearable data that are indicative of these events as learned in the training set.

While one or more embodiments of the invention have been described, the invention is not limited to these embodiments and the scope of the invention is defined by reference to the following claims.

1. A wearable sensor device, for sensing and recording data representative of events of memory lapses and function, comprising:
a wearable sensor device;
at least one gesture sensor in the wearable sensor device capable of sensing a gesture by the wearer, the gesture being representative of events of memory lapses and function;
at least one vital sign sensor for sensing at least one vital sign condition being experienced by a wearer of the device;
a memory for storing gesture data representing the sensed data from the gesture sensor, and for storing vital sign data sensed by the vital sign sensor;
wherein the gesture data and vital sign data is adapted for transmission to a computation unit for analyzing the gesture data and vital sign data, comparing it to a reference database of normative data of age-matched subjects and for producing diagnosis data which predicts onset of cognitive impairment related diseases.

2. The device according to claim 1, wherein the gesture sensor detects at least one of a tap, a tap sequence, an audio signal, a video signal, a hand gesture, a head movement gesture, an audible trigger, and an EEG trigger.

3. The device according to claim 1, wherein the vital sign sensor detects at least one of heart rate, blood pressure, perspiration, EEG temperature and blood oxygen level.

4. The device according to claim 1, further including at least one activity sensor for detecting at least one of sleep exercise, motion and mobility.

5. The device according to claim 1, wherein the device communicates the gesture and vital sign data to a cloud server.

6. The device according to claim 5, wherein the device communicates the gesture and vital sign data through a router to a cloud server.

7. The device according to claim 5, wherein the device communicates the gesture and vital sign data through a Bluetooth low energy (BLE) device to a cloud server.

8. The device according to claim 5, wherein the device communicates the gesture and vital sign data through a charging base to cloud server.

9. The device according to claim 8, wherein the device communicates the gesture and vital sign data through a charging base and router to a cloud server.

10. The device according to claim 5, wherein the device communicates the gesture and vital sign data continuously in real time.

11. The device according to claim 5, wherein the device communicates the gesture and vital sign data in batches.

12. The device according to claim 1, wherein the computation unit calculates a risk factor score based on at least one of frequency of memory lapses, time and quality of sleep, amount and duration of exercise, mobility, heart rate, blood pressure, perspiration and diet.

13. The device according to claim 1, wherein the computation unit predicts onset of cognitive impairment related diseased by analyzing the circumstances under which the memory lapses and function occurred, and determining the type of memory lapse, including one or more components of cognitive or function.

14. The device according to claim 13, wherein the computation unit predicts onset by analyzing gesture and vital sign data for a time period offset in time from a gesture representative of a memory lapse event.

15. The device according to claim 14, wherein the time period offset includes a time period which precedes a gesture representative of a memory lapse event.

16. The device according to claim 14, wherein the time period offset includes a time period which is subsequent to a gesture representative of a memory lapse event.

17. The device according to claim 1, wherein the sensor is an audio sensor and wherein the gesture data is audio data.

18. The device according to claim 1, wherein the sensor is a video sensor and wherein the gesture data is video data of the subject wearing the wearable device.

19. The device according to claim 17, wherein the computation unit further includes a speech recognition unit.

20. The device according to claim 1, wherein the computation unit receives gesture data and vital sign data from a plurality of users wearing a wearable device, and uses the combined data to generate population risk factors.

21. The device according to claim 20, wherein the combined data is used to generate population risk factors for advancing disease.

22. The device according to claim 1, wherein the computation unit compares the gesture and vital sign data to previously obtained baseline data.

23. A method for sensing and recording data representative of events of memory lapses and function, comprising:
providing a wearable sensor device worn by a subject;
sensing a gesture by the wearer using a gesture sensor in the wearable sensor device, the gesture being representative of events of memory lapses and function;
sensing at least one vital sign condition being experienced by a wearer of the device using a vital sign sensor in the wearable sensor device;
storing vital sign gesture data representing the sensed data from the gesture sensor, and storing vital sign data sensed by the vital sign sensor;
transmitting the gesture data and vital sign data to a computation unit;
comparing the gesture data and vital sign data to a reference database of normative data of age-matched subjects; and
producing diagnosis data which predicts onset of cognitive impairment related diseases of the subject.

24. The method according to claim 23, wherein the sensing step detects at least one of a tap, a tap sequence, an audio signal, a video signal, a hand gesture, a head movement gesture, an audible trigger, and an EEG trigger.

25. The method according to claim 23, wherein the vital sign sensor detects at least one of heart rate, blood pressure, perspiration, EEG temperature and blood oxygen level.

26. The method according to claim 23, including detecting at least one of sleep exercise, motion and mobility of the subject, and providing activity data.

27. The method according to claim 23, including communicating the gesture and vital sign data to a cloud server.

28. The method according to claim 27, wherein the device communicates the gesture and vital sign data through a router to a cloud server.

29. The method according to claim 27, wherein the device communicates the gesture and vital sign data through a Bluetooth low energy (BLE) device to a cloud server.

30. The method according to claim 27, wherein the device communicates the gesture and vital sign data through a charging base to cloud server.

31. The method according to claim 29, wherein the device communicates the gesture and vital sign data through a charging base and router to a cloud server.

32. The method according to claim 27, wherein the device communicates the gesture and vital sign data continuously in real time.

33. The device according to claim 27, wherein the device communicates the gesture and vital sign data in batches.

34. The device according to claim 23, wherein the computation unit calculates a risk factor score based on at least one of frequency of memory lapses, time and quality of sleep, amount and duration of exercise, mobility, heart rate, blood pressure, perspiration and diet.

35. The device according to claim 23, wherein the computation unit predicts onset of cognitive impairment related diseased by analyzing the circumstances under which the memory lapses and function occurred, and determining the type of memory lapse, including one or more components of cognitive or function.

36. The method according to claim 35, including predicting onset by analyzing gesture and vital sign data for a time period offset in time from a gesture representative of a memory lapse event.

37. The method according to claim 36, including analyzing gesture and vital sign data in a time period which precedes a gesture representative of a memory lapse event.

38. The method according to claim 36, including analyzing gesture and vital sign data in a time period which is subsequent to a gesture representative of a memory lapse event.

39. The method according to claim 36, wherein the gesture data is at least one of audio data and video data of the subject wearing the wearable device.

40. The method according to claim 23, wherein the computation unit further includes a speech recognition unit for recognizing speech.

41. The method according to claim 23, including receiving gesture data and vital sign data from a plurality of users wearing a wearable device, and using the combined data to generate population risk factors.

42. The method according to claim 42, including generating population risk factors for advancing disease.

43. The method according to claim 23, including comparing the gesture and vital sign data to previously obtained baseline data.

44. A non-transitory storage medium for storing instructions for sensing and recording data representative of events of memory lapses and function of a subject wearing a wearable sensing device, wherein the instructions perform the steps of:
sensing a gesture by the wearer using a gesture sensor in the wearable sensor device, the gesture being representative of events of memory lapses and function;
sensing at least one vital sign condition being experienced by a wearer of the device using a vital sign sensor in the wearable sensor device; and
storing vital sign gesture data representing the sensed data from the gesture sensor, and storing vital sign data sensed by the vital sign sensor.

45. The storage medium of claim 44, which further includes instructions for:
transmitting the gesture data and vital sign data to a computation unit;
comparing the gesture data and vital sign data to a reference database of normative data of age-matched subjects; and
producing diagnosis data which predicts onset of cognitive impairment related diseases of the subject.
